(19) United States
(12) Patent Application Publication
(10) Pub. No.: US 2024/0158329 A1
(43) Pub. Date: May 16, 2024

(54) METHOD FOR PURIFYING VANILLIN OR DERIVATIVES THEREOF OBTAINED BY A BIOTECHNOLOGICAL METHOD

(71) Applicant: RHODIA OPERATIONS FRANCE, Lyon (FR)

(72) Inventors: Talsida Zougmore, Francheville (FR); Rachid Djillali, Lyon (FR); Laurent Garel, Lyon (FR)

(73) Assignee: SPECIALTY OPERATIONS FRANCE, Lyon (FR)

(21) Appl. No.: 18/550,931

(22) PCT Filed: Mar. 15, 2022
(86) PCT No.: PCT/EP2022/056761

§ 371 Date: Sep. 15, 2023

(30) Foreign Application Priority Data
Mar. 15, 2021 (FR) FR2102537

(51) Int. Cl. C07C 45/81; C07C 45/78; C07C 45/80

(52) U.S. Cl. CPC C07C 45/81; C07C 45/786; C07C 45/80

(57) ABSTRACT

The present invention relates to a process for purifying a fermentation must (M), obtained via a biotechnological process, comprising biomass and vanillin or derivatives thereof in aqueous solution, for the preparation of a crystallized vanillin or derivatives thereof, characterized in that, throughout the purification process, the vanillin or derivatives thereof in protonated or salified form remain in aqueous solution.

### FIELD OF THE INVENTION

The present invention relates to a process for purifying vanillin or derivatives thereof obtained via a biotechnological process.

### PRIOR ART

Vanillin may be obtained by various methods known to those skilled in the art, notably by the following two routes:
a “natural” route based on a biotechnological process notably comprising the cultivation of a microorganism that is capable of enabling the biotransformation of a fermentation substrate into vanillin. Such a process, in which the fermentation substrate is ferulic acid, is notably known from patent application EP 0885968. Patent U.S. Pat. No. 5,017,388 describes a process in which the fermentation substrate is eugenol and/or isoeugenol. These processes lead to the preparation of a vanillin known as natural vanillin.
a “synthetic” route comprising conventional chemical reactions starting from guaiacol and not involving microorganisms. This process leads to the preparation of a vanillin known as synthetic vanillin.

Finally, vanillin can also be prepared via a “biobased” route, in which vanillin is derived from lignin. In particular, mention may be made of U.S. Pat. No. 2,745,796, DE 1132113 and the article entitled “Preparation of lignin from wood dust as vanillin source and comparison of different extraction methods” by Azadbakht et al. in International Journal of Biology and Biotechnology, 2004, vol. 1, No. 4, pages 535-537.

At the present time, natural vanillin can be purified via the process described in patent application EP 2791098, which includes a step of liquid/liquid extraction of impurities with a pKa higher than that of vanillin. The yield for this process is good, generally greater than 80%, but in order to obtain improved organoleptic properties such as the odor and/or color of vanillin, several additional purification steps are required, thus causing the overall yield of this process to fall. The overall energy efficiency of this process is also degraded due to the use of large amounts of solvents.

International patent application WO 2014/114590 also describes a process for purifying natural vanillin. This process consists in evaporating natural vanillin, which may be performed by distillation or by vacuum evaporation of molten vanillin. This process is capable of producing very pure natural vanillin, in good yield, with a device that is simple to use and which operates continuously so as to be compatible with industrial processes. However, such a process may be difficult to implement due to the number and size of the items of equipment required.

Moreover, vanillin or derivatives thereof obtained via a biotechnological process may contain certain impurities with boiling points very close to that of vanillin or derivatives thereof.

Thus, it is necessary to size the equipment suitably so as to enable efficient separation of vanillin or derivatives thereof from these products. This usually involves extending the residence time in the distillation equipment, which may generate new impurities due to the high-temperature instability of vanillin or derivatives thereof and/or impurities.

For this reason, it would be advantageous to have an improved process relative to those proposed in the prior art, notably in terms of environmental and/or energy impact, while at the same time improving the overall purification yield and also the yield of vanillin or derivatives thereof. It is also important that the process for purifying vanillin or derivatives thereof allows the production of a vanillin or derivatives thereof whose organoleptic properties are preserved notably in terms of taste, color and/or odor.

### BRIEF DESCRIPTION

The present invention relates to a process for purifying a fermentation must (M), obtained via a biotechnological process, comprising biomass and vanillin or derivatives thereof in aqueous solution, for the preparation of crystallized vanillin or derivatives thereof, characterized in that throughout the purification process, vanillin or derivatives thereof in protonated or salified form remain in aqueous solution, comprising:
a step (a) of separating the biomass of a fermentation must (M) from an aqueous stream (A1) comprising vanillin or derivatives thereof, and
at least one step in which the aqueous stream (A1) is subjected to a filtration step by nanofiltration or reverse osmosis, characterized in that the reverse osmosis has a cut-off limit of less than or equal to 100 Da and the nanofiltration has a cut-off limit of less than or equal to 400 Da, preferably between 100 and 250 Da.

### DESCRIPTION OF THE FIGS

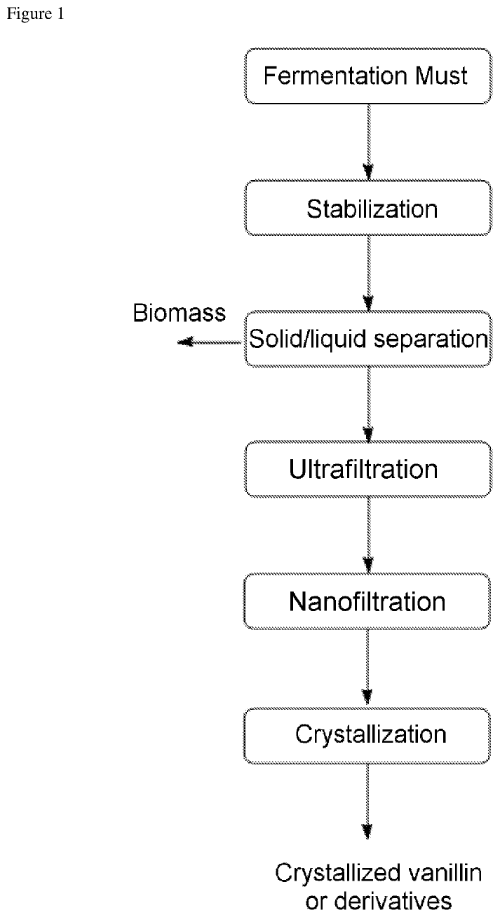

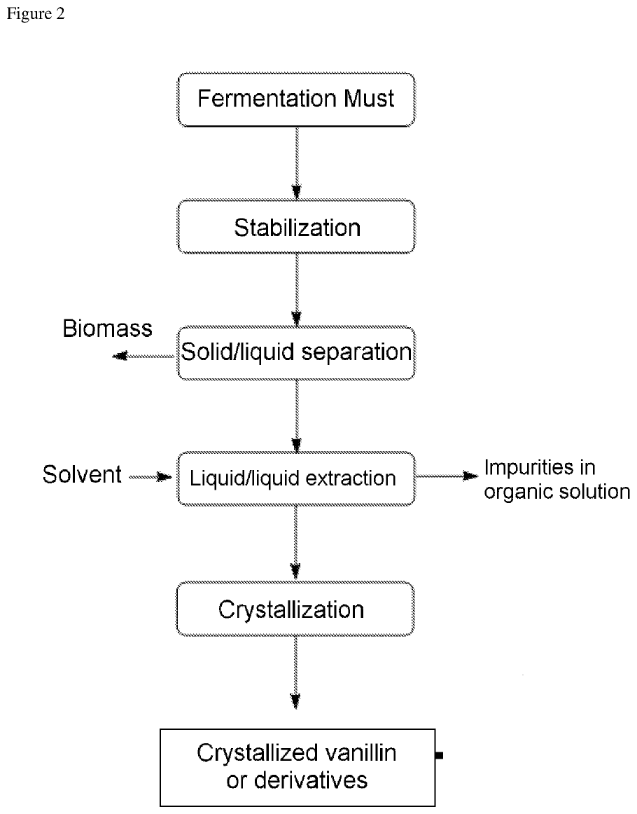

FIGS. 1 and 2 schematically show various processes for purifying vanillin or derivatives thereof obtained via a biotechnological process according to the present invention.

### DETAILED DESCRIPTION

In the context of the present invention, and unless otherwise indicated, the expression “between . . . and . . . ” includes the limits.

In the present invention, the term “natural vanillin” denotes a vanillin obtained via a biotechnological process. Thus, a process for preparing natural vanillin denotes herein a biotechnological process comprising the cultivation of a microorganism that is capable of enabling the transformation of a fermentation substrate into vanillin. The microorganism may be of wild-type origin or may be a genetically modified microorganism (GMM) obtained via molecular biology. Very preferentially, it may be a ferulic acid fermentation process, such as that described in patent application EP 0885968. According to a particular aspect, vanillin may be produced via a glucose or protocatechuic acid fermentation process as described in patent application WO 2013/022881.

In the context of the present invention, the term “vanillin derivative” refers to any compound that may be derived from vanillin and in particular to vanillin in salified form or glucovanillin.

In the context of the present invention, the term “vanillyl alcohol derivative” refers to any compound that may be derived from vanillyl alcohol and in particular to vanillyl alcohol in salified form or to vanillyl alcohol glucoside.

In the context of the present invention, the term “crystallization” refers to a process in which a substance passes into the solid state via a physical process, notably such as lowering the temperature.

In the context of the present invention, the term “precipitation” refers to a process in which a substance passes into the solid state via a chemical transformation, notably such as protonation through a change in pH.

In the context of the present invention, the term “vanillin” or “vanillin in protonated form” denotes 4-hydroxy-3-methoxybenzaldehyde.

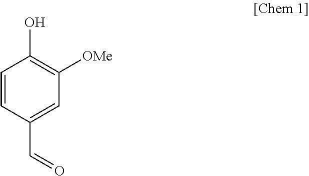 [SMILES: COc1cc(C=O)ccc1O]

The term “vanillin in salified form” refers to the salts of 4-hydroxy-3-methoxybenzaldehyde, the formula of which is defined below, in which the Metal is chosen from sodium and potassium.

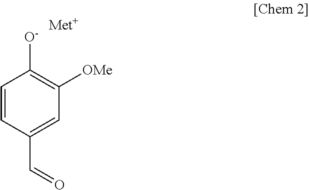

The present invention relates to a process for purifying a fermentation must (M), obtained via a biotechnological process, comprising biomass and vanillin or derivatives thereof in aqueous solution, for the preparation of a crystallized vanillin or derivatives thereof, characterized in that, throughout the purification process, the vanillin or derivatives thereof in protonated or salified form remain in aqueous solution, comprising:
a step (a) of separating the biomass of a fermentation must (M) from an aqueous stream (A1) comprising vanillin or derivatives thereof, and
at least one step in which the aqueous stream (A1) is subjected to a filtration step by nanofiltration or reverse osmosis, characterized in that the reverse osmosis has a cut-off limit of less than or equal to 100 Da and the nanofiltration has a cut-off limit of less than or equal to 400 Da, preferably between 100 and 250 Da.

According to the present invention, the process for purifying a fermentation must (M) comprises a step (a) of separating the biomass of a fermentation must (M) from an aqueous stream (A1) comprising vanillin or derivatives thereof.

This step is a step of separating a solid phase: the biomass, from a liquid phase. The liquid phase obtained on conclusion of the biomass separation step is an aqueous stream (A1) comprising vanillin or derivatives thereof, in salified or protonated form.

According to one aspect, the biomass separation step may be performed by filtration such as frontal filtration or tangential filtration, in particular membrane filtration, such as microfiltration, ultrafiltration, nanofiltration, or reverse osmosis. Membrane filtration may be performed by concentration or diafiltration. Membrane filtration may also be performed by concentration and diafiltration. When the filtration is performed by diafiltration, it is possible to use water or a recycle stream resulting from the process according to the present invention, for example the recycle stream resulting from the filtration. The recycle stream may contain vanillin or derivatives thereof in protonated or salified form. Advantageously, when the biomass separation step is frontal filtration, adjuvants may be added so as to improve the filtration efficacy.

According to another aspect of the present invention, the biomass separation step may be performed by centrifugation, notably on a disc or plate centrifuge. This embodiment may prove advantageous when it is performed upstream of membrane filtration, as it allows the service life of the membranes used to be extended.

In order to improve the solid/liquid separation efficacy, it is possible to perform several solid/liquid separation steps, notably to remove the smallest solid particles.

According to a particular aspect, the biomass separation step may be performed with one or more microfiltration membranes, in particular having a cut-off limit of about 0.2 μm, followed by one or more ultrafiltrations having a cut-off limit smaller than that of the microfiltration. In this configuration, the vanillin or derivatives thereof are dissolved in the aqueous phase and the biomass is retained by the membranes. Advantageously, ultrafiltration also allows the separation of molecules dissolved in the fermentation must.

In order to improve the yield of vanillin or derivatives thereof in the aqueous phase, a solvent, preferably water, is added during the filtration step. In general, the amount of solvent added is between 0.5 and 5 volume equivalents of fermentation must.

According to one aspect, microfiltration, ultrafiltration or diafiltration may be coupled with a nanofiltration or reverse osmosis step. Nanofiltration allows the concentration of vanillin in the retentate of the nanofiltration step to be increased, while the water passes through the membrane (permeate of the nanofiltration step). The permeate from this nanofiltration step may advantageously be recycled. In general, reverse osmosis has a cut-off limit of less than or equal to 100 Da. In general, the reverse osmosis membrane allows the retentate to retain at least 97% NaCl, preferably at least 98% NaCl, very preferentially at least 99%, and even more preferentially at least 99.5% NaCl. In general, nanofiltration has a cut-off limit of less than or equal to 400 Da. In general, nanofiltration has a cut-off limit of greater than or equal to 100 Da, for example a cut-off limit of between 100 and 250 Da. Nanofiltration or reverse osmosis may notably be operated in concentration. In general, the pH of the aqueous solution of vanillin or derivatives thereof is adapted to be compatible with the type of nanofiltration or reverse osmosis membrane used in the process of the present invention. The pH may be between 1 and 11. The pH may notably be suitable for retaining some, preferably most, of the amount of vanillin or derivatives thereof in the retentate. Nanofiltration is generally operated under pressure, usually between 10 and 50 bar, preferably between 10 and 30 bar. Reverse osmosis is generally operated under pressure, the pressure generally being greater than or equal to 30 bar, preferably between 30 and 60 bar, preferably between 40 and 60 bar. The nanofiltration or reverse osmosis membrane may advantageously be a ceramic or polymeric membrane, preferably of the polyamide, polypiperazine-amide, aromatic polyamide or cellulose acetate type.

Step (a0)

Prior to this step of separating the biomass from the aqueous solution of vanillin or derivatives thereof, the fermentation must may be stabilized. Thus, the process of the present invention may optionally comprise a step (a0) of stabilizing the fermentation must (M). Step (a) of the process according to the present invention may be performed on a stabilized fermentation must (M).

In the context of the present invention, the term “stabilization” refers to any method for preventing the degradation, notably by reduction, of vanillin or derivatives thereof between the end of fermentation and the purification process.

According to a first aspect, stabilization may be performed by the addition of at least one compound. The compound is preferentially chosen from sodium benzoate, ascorbic acid and salts thereof, potassium, calcium or sodium sorbate, zinc sulfate, propanoic acid, acetic acid or salts thereof, or sodium diacetate. Preferably, the amount of compound added is between 0.2 g/L and 6 g/L.

According to another aspect, stabilization may be performed by changing the temperature of the fermentation must. In general, the temperature is controlled so as to achieve a temperature of between 15° C. and 23° C., preferably between 18° C. and 21° C.

According to another aspect, stabilization may be performed by changing the pH of the fermentation must. In general, the pH is controlled so as to achieve a pH of less than or equal to 7.5, preferably less than or equal to 7, very preferentially less than or equal to 6.8. In general, the pH is controlled to achieve a pH of greater than or equal to 5.0, preferably greater than or equal to 6.

According to another aspect, the fermentation must may also be pasteurized. In general, the fermentation must is then heated to a temperature of between 50° C. and 90° C., preferably between 60° C. and 80° C. Heating is generally maintained for between 10 min and 120 min, preferably between 15 min and 45 min, for example for 20 min.

According to another aspect, the fermentation must may be stabilized by ultrasound. In general, ultrasound is emitted into the fermentation must for a period of between 10 min and 120 min.

According to a particular aspect, these aspects may be performed separately or in combination; thus, by way of nonlimiting example, it is possible to modify the temperature, the pH and to add a compound under the conditions described above.

Among these methods, methods without adding compounds are advantageous: without wishing to be bound by any theory, they allow possible deleterious effects on vanillin or derivatives thereof, notably in terms of odor or color, to be avoided.

However, the addition of certain compounds chosen for their compatibility with vanillin or derivatives thereof and the absence of side effects on vanillin or derivatives thereof may prove particularly advantageous in facilitating the subsequent process for purifying vanillin or derivatives thereof.

According to one aspect of the present invention, the aqueous stream (A1) comprising vanillin or derivatives thereof is subjected to at least one filtration step such as frontal filtration or tangential filtration, in particular membrane filtration, such as microfiltration, ultrafiltration, nanofiltration, or reverse osmosis. In general, reverse osmosis has a cut-off limit of less than or equal to 100 Da. In general, the reverse osmosis membrane allows at least 97% NaCl, preferably at least 98% NaCl, very preferentially at least 99%, and even more preferentially at least 99.5% NaCl to be retained in the retentate. In general, the nanofiltration has a cut-off limit of less than or equal to 400 Da. In general, nanofiltration has a cut-off limit of greater than or equal to 100 Da, for example a cut-off limit of between 100 and 250 Da. The membrane filtration may be operated in concentration or in diafiltration. The membrane filtration may also be operated in concentration and in diafiltration. When the filtration is performed by diafiltration, it is possible to use water or a recycle stream resulting from the filtration. The recycle stream may contain vanillin or derivatives thereof in protonated or salified form. In general, the pH of the aqueous solution of vanillin or derivatives thereof is adapted to be compatible with the type of nanofiltration or reverse osmosis membrane used in the process of the present invention. The pH may be between 1 and 11. The pH may notably be suitable for retaining some, preferably most, of the amount of vanillin or derivatives thereof in the retentate. Nanofiltration is generally operated under pressure, usually between 10 and 50 bar, preferably between 10 and 30 bar. Reverse osmosis is generally operated under pressure, the pressure generally being greater than or equal to 30 bar, preferably between 30 and 60 bar, preferably between 40 and 60 bar. The nanofiltration or reverse osmosis membrane may advantageously be a ceramic or polymeric membrane, preferably of the polyamide, polypiperazine-amide, aromatic polyamide or cellulose acetate type.

The use of a nanofiltration membrane with a cut-off limit of less than or equal to 400 Da or a reverse osmosis membrane with a cut-off limit of less than or equal to 100 Da, or a reverse osmosis membrane that is capable of retaining at least 97% NaCl, preferably at least 98% NaCl, very preferentially at least 99%, and even more preferentially at least 99.5% NaCl in the retentate, is particularly suitable for allowing a good yield of vanillin or derivatives thereof (excluding recycle), coupled with the use of minimum pressure. Thus, the energy costs associated with the purification process according to the present invention are kept under control.

In general, the filtration process may be performed using a ceramic membrane, preferably with a cut-off limit of about 0.2 μm.

The filtrate obtained is an aqueous stream (A2) comprising vanillin or derivatives thereof in protonated or salified form, in which impurities have been separated out.

The filtrate may then be subjected to at least one nanofiltration or reverse osmosis step, so as to concentrate the aqueous stream (A2) comprising vanillin. Nanofiltration allows the concentration of vanillin or derivatives thereof in the retentate of the nanofiltration step to be increased, while the water passes through the membrane (permeate of the nanofiltration step). The permeate from this nanofiltration step may advantageously be recycled. In general, reverse osmosis has a cut-off limit of less than or equal to 100 Da. In general, the reverse osmosis membrane allows the retentate to retain at least 97% NaCl, preferably at least 98% NaCl, very preferentially at least 99%, and even more preferentially at least 99.5% NaCl. In general, nanofiltration has a cut-off limit of less than or equal to 400 Da. In general, nanofiltration has a cut-off limit of greater than or equal to 100 Da, for example a cut-off limit of between 100 and 250 Da. Membrane filtration may be performed by concentration or diafiltration. Membrane filtration may also be performed by concentration and diafiltration. When the filtration is performed by diafiltration, it is possible to use water or a recycle stream resulting from the filtration. The recycle stream may contain vanillin or derivatives thereof in protonated or salified form. In general, the pH of the aqueous solution of vanillin or derivatives thereof is adapted to be compatible with the type of nanofiltration or reverse osmosis membrane used in the process of the present invention. The pH may be between 1 and 11. The pH may notably be suitable for retaining some, preferably most, of the amount of vanillin or derivatives thereof in the retentate. Nanofiltration is generally operated under pressure, usually between 10 and 50 bar, preferably between 10 and 30 bar. Reverse osmosis is generally operated under pressure, the pressure generally being greater than or equal to 30 bar, preferably between 30 and 60 bar, preferably between 40 and 60 bar. The nanofiltration or reverse osmosis membrane may advantageously be a ceramic or polymeric membrane, preferably of the polyamide, polypiperazine-amide, aromatic polyamide or cellulose acetate type.

The process of the present invention includes at least one step of crystallizing vanillin or derivatives thereof contained in the aqueous stream (A2).

The crystallization step allows the purification of an aqueous stream (A2) comprising vanillin or derivatives thereof. Preferably, the aqueous stream (A2) has a purity of between 85% and 99%, preferably greater than or equal to 90%, very preferentially greater than or equal to 95%. Advantageously, the crystallization step allows the production of purified vanillin or derivatives thereof with a purity of greater than or equal to 95%, preferably greater than or equal to 97%, very preferentially greater than or equal to 99%. Vanillin or derivatives thereof with a purity of between 85% and 99% may also comprise at least one other compound chosen from vanillyl alcohol or derivatives thereof, vanillic acid, guaiacol, acetovanillone, 4-((4-hydroxy-3-methoxybenzyl)oxy)-3-methoxybenzaldehyde and 4-hydroxy-3-(4-hydroxy-3-methoxybenzyl)-5-methoxybenzaldehyde.

The crystallization is generally performed in an alcoholic solution. Preferably, the solvent used for the crystallization may be a water-soluble alcohol, preferably ethanol. The solvent used for the crystallization may be a water/alcohol mixture. In general, the amount of alcohol is between 2% and 40% by mass, preferably between 5% and 35% by mass, and very preferentially between 15% and 25% by mass. In general, during crystallization, the concentration of vanillin or derivatives thereof at the start of crystallization is between 5% and 60% by weight, preferably between 10% and 50% by weight, advantageously between 15% and 35% by weight, and even more preferentially between 15% and 25%. Very advantageously, crystallization allows the separation of vanillin or derivatives thereof from the impurities contained in the aqueous stream (A2), and this separation is advantageously performed without degradation of the vanillin or derivatives thereof. Crystallization takes place at a temperature of between 0° C. and 50° C. The crystallization yield is generally greater than or equal to 80%.

According to another embodiment, the crystallization may be performed in ethanol. The aqueous stream (A2) is evaporated, and ethanol is then added so as to crystallize the vanillin.

Advantageously, the process of the present invention does not require any organic solvent, except for the crystallization solvent.

Alternatively, the aqueous stream (A2) may be subjected to a precipitation step. Precipitation of vanillin or derivatives thereof is generally performed at a temperature of between 0° C. and 6° C. During precipitation, the pH of the aqueous stream (A2) may be adjusted to a value of between 5 and 8. A vanillin precipitate (P) is obtained.

The precipitate (P) may be subjected to at least one crystallization step as described above.

The vanillin or derivatives thereof obtained on conclusion of the precipitation and/or crystallization step generally have a color in ethanolic solution at 10% by weight of less than or equal to 150 Hazen, preferably less than or equal to 100 Hazen, and very preferentially less than or equal to 50 Hazen.

According to another aspect of the purification process, the aqueous stream (A1) comprising vanillin or derivatives thereof, in salified or protonated form, is subjected to a liquid/liquid extraction step. During the liquid/liquid extraction step, the vanillin or derivatives thereof in salified form are washed in an aqueous stream (A2). Impurities contained in the aqueous stream (A1) are extracted in organic solution (01).

In general, the pH of the aqueous solution is greater than 7, preferably greater than 7.5, very preferentially greater than 8.

In general, the extraction solvent is chosen for its capacity for dissolving the impurities contained in the aqueous stream (A1); advantageously, the solvent may be of biobased origin.

Preferably, the solubility of vanillin or derivatives thereof in protonated or salified form is better in water than in the extraction solvent. According to one aspect, the chosen solvent is compatible with food industry standards, notably FEMA GRAS, and is water-immiscible. Preferably, the solvent is chosen from methyl ethyl ketone, methyl isobutyl ketone, ethyl acetate, isopropyl acetate or mixtures thereof.

The liquid/liquid extraction step may be a discontinuous liquid/liquid extraction. In order to maximize the amount of vanillin or derivatives thereof obtained in the aqueous phase (A2), the volume ratio of solvent relative to the aqueous stream (A1) comprising vanillin or derivatives thereof is between 1:5 and 5:1, preferably between 1:2 and 5:1, preferentially between 1:1 and 5:1, preferably between 1.5:1 and 3:1.

According to another aspect, the liquid/liquid extraction step may be performed continuously. In general, the volume ratio of solvent relative to the aqueous stream (A1) comprising vanillin or derivatives thereof is between 5:1 and 1:2, preferably between 3:1 and 1:1.

The yield of vanillin or derivatives thereof from the liquid/liquid extraction step is generally greater than or equal to 95%, preferably greater than or equal to 97%, very preferentially greater than or equal to 98%.

According to a particular aspect, the biomass separation and liquid/liquid extraction steps may be performed simultaneously. This particular aspect allows vanillin or derivatives thereof in protonated or salified form in the aqueous stream (A2) to be washed from the fermentation must. The biomass is then separated from the two-phase system. This process is advantageous in that the loss of vanillin or derivatives thereof in the biomass is reduced. This separation step may notably be performed continuously by centrifugation, in particular using a two-phase or three-phase centrifuge or a countercurrent extractor, preferably a three-phase centrifuge.

The concentration (C) of the aqueous stream (A2), comprising vanillin or derivatives thereof, obtained on conclusion of the liquid/liquid extraction step is generally between 0.1% by weight and 10% by weight.

The aqueous stream (A2) may then be subjected to a crystallization step as described previously.

Prior to the crystallization step, the aqueous stream (A2) comprising vanillin or derivatives thereof may be concentrated so that the concentration of vanillin or derivatives thereof in the aqueous stream is between 5% and 60% by weight.

Alternatively, the aqueous stream (A2) may be subjected to a precipitation step. Precipitation of vanillin or derivatives thereof is generally performed at a temperature of between 0° C. and 6° C. During precipitation, the pH of the aqueous stream (A2) may be adjusted to a value of between 5 and 8. A precipitate (P) of vanillin or derivatives thereof is obtained.

The precipitate (P) may be subjected to at least one crystallization step as described above.

In the context of the present invention, prior to the crystallization and/or precipitation steps, the aqueous stream (A2) may optionally be treated on charcoal or resin, as described notably in patent application WO 2011/039331.

Advantageously, the vanillin or derivatives thereof, obtained on conclusion of the process according to the present invention, generally have a color in ethanolic solution at 10% by weight of less than or equal to 150 Hazen, preferably less than or equal to 100 Hazen, and very preferentially less than or equal to 50 Hazen. Moreover, the vanillin or derivatives thereof obtained on conclusion of the purification process according to the present invention have compliant organoleptic properties.

### EXAMPLES

### Example 1

A stabilized fermentation must as defined in Table 1 is subjected to a purification process as described hereinbelow.

TABLE 1
Composition of the fermentation must

| | | |
|---|---|---|
| Biomass | 1.5% | by weight |
| Vanillin | 2% | by weight |
| Water | 95% | by weight |
| Other compounds | 1.5% | by weight |

The biomass is separated from the aqueous phase by tangential diafiltration on a ceramic membrane with a pore diameter of 0.2 μm. The addition of 2 dia-volumes allows a vanillin yield of >95% to be obtained.

The resulting aqueous solution, separated from the biomass, is salified to pH=8.5 and then concentrated by reverse osmosis. This last vanillin solution, in salified and concentrated form, is washed with its equivalent volume of isopropyl acetate so as to remove any non-salified organic compounds. The salified vanillin is then precipitated by reducing the pH to 5, filtered off and washed with water before being dried and ground.

The purity of the vanillin obtained is 98% and it has good organoleptic properties.

### Example 2

A fermentation must as defined in Example 1 is subjected to a biomass separation step as described in Example 1 to give an aqueous solution.

The aqueous solution is then subjected to a nanofiltration
step under the conditions below:

| Reference | Membrane type | Cut-off threshold | Vanillin yield |
|---|---|---|---|
| 1 | Polyamide | 400 Da | 88% |
| 2 | Polyamide | 250 Da | 93% |
| 3 (Comparative) | Polyamide | 800 Da | <70% |

1. A process for purifying a fermentation must (M), obtained via a biotechnological process, comprising biomass and vanillin or derivatives thereof in aqueous solution, for the preparation of a crystallized vanillin or derivatives thereof, characterized in that, throughout the purification process, the vanillin or derivatives thereof in protonated or salified form remain in aqueous solution, the process comprising:
a step of separating the biomass of a fermentation must (M) from an aqueous stream (A1) comprising vanillin or derivatives thereof, and
at least one step of filtering the aqueous stream (A1) by nanofiltration or reverse osmosis, characterized in that the reverse osmosis has a cut-off limit of less than or equal to 100 Da and the nanofiltration has a cut-off limit of less than or equal to 400 Da.

2. The purification process of claim 1, characterized in that the nanofiltration or the reverse osmosis comprise a membrane, in which the membrane is a ceramic or polymeric membrane.

3. The purification process of claim 1, characterized in that the pH is between 1 and 11.

4. The purification process of claim 1, characterized in that the pressure during nanofiltration is between 10 and 50 bar, or is greater than or equal to 30 bar in reverse osmosis.

5. The purification process of claim 1, further comprising a step (a0) of stabilizing the fermentation must (M).

6. The purification process of claim 1, characterized in that the aqueous stream (A1) comprising vanillin or derivatives thereof is subjected to at least one filtration step, thereby allowing an aqueous stream (A2) comprising vanillin or derivatives thereof to be obtained, in protonated or salified form.

7. The purification process of claim 1, characterized in that the aqueous stream (A1) comprising vanillin or derivatives thereof, in salified form, is subjected to a liquid/liquid extraction step, thereby allowing an aqueous stream (A2) comprising vanillin or derivatives thereof, in protonated or salified form, to be obtained.

8. The purification process of claim 6, characterized in that said process comprises at least one step of crystallizing the vanillin or derivatives thereof contained in the aqueous stream (A2).

9. The purification process of claim 6, characterized in that said process comprises at least one step of precipitating the vanillin or derivatives thereof contained in the aqueous stream (A2).

10. The purification process of claim 1, characterized in that the vanillin or derivatives thereof obtained have a color in ethanolic solution at 10% by weight of less than or equal to 150 Hazen.

11. The purification process of claim 1, in which the cut-off limit of the nanofiltration is between 100 and 250 Da.

12. The purification process of claim 2, in which the polymeric membrane is one or more selected from a polyamide, a polypiperazine-amide, an aromatic polyamide, or a cellulose acetate type membrane.

13. The purification process of claim 6, in which the filtration step comprises frontal filtration or tangential filtration.

14. The purification process of claim 6, in which the filtration step comprises membrane filtration.

15. The purification process of claim 14, in which the membrane filtration is microfiltration or ultrafiltration.

16. The purification process of claim 8, in which the at least one step of crystallizing the vanillin or derivatives thereof contained in the aqueous stream (A2) is performed in an alcoholic solution.

17. The purification process of claim 1, characterized in that the vanillin or derivatives thereof obtained have a color in ethanolic solution at 10% by weight of less than or equal to 100 Hazen.

18. The purification process of claim 1, characterized in that the vanillin or derivatives thereof obtained have a color in ethanolic solution at 10% by weight of less than or equal to less than or equal to 50 Hazen.
